SEQUENCE LISTING (5 sequences)

SEQ ID NO: 1 - DNA, 3851 bp, Ralstonia eutropha
  source 1..3851: mol_type = genomic DNA; strain = ATCC 17699
atggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatgacgcttgcatgagtgccggcgtgcgtcatgcacggcgccggcaggcctgcaggttccctcccgtttccattgaaaggactacacaatgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataaggaaggggttttccggggccgcgcgcggttggcgcggacccggcgacgataacgaagccaatcaaggagtggacatgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctga

SEQ ID NO: 2 - DNA, 1371 bp, Methylophaga aminisulfidivorans
  source 1..1371: mol_type = genomic DNA; strain = MP 54_1
atggcaactcgtattgcgatacttggtgcaggcccaagtggtatggcacaactcagagcattccaatccgcccaggaaaaaggtgctgagatccctgaactcgtttgttttgaaaaacaagctgattggggcggccagtggaattacacatggcgcactggtttagatgaaaatggcgaacctgttcatagcagtatgtatcgctatctgtggtcaaacggcccgaaagaatgtcttgaatttgctgattacacgtttgacgaacactttggtaagcccatcgcctcttatccaccccgtgaagtcttatgggactatattaaaggccgtgttgaaaaagccggcgtcagaaaatatatccgttttaataccgctgttcgtcatgttgaattcaacgaagacagccaaacttttaccgttaccgtgcaggaccatactactgacacaatttactctgaagagtttgactatgttgtctgttgtaccggtcacttctcaacaccttacgtgcctgaatttgaaggctttgaaaaatttggtggccgcattctgcatgcccatgacttccgtgacgcattagaatttaaagacaaaactgtattactggtcggcagcagttactcagctgaagatatcggctcacaatgttataaatacggcgcgaaaaaactgatcagctgctaccgtaccgcaccgatgggttataaatggcctgaaaactgggatgaaagacccaacctggttcgtgttgatactgaaaacgcttattttgccgatggttcatcagaaaaagtcgatgcgattattctgtgtaccggttatatccatcacttccccttcctcaatgacgatctgcgtctggtcaccaataaccgtttatggccgctcaacctttataaaggcgtggtgtgggaagataatccaaaattcttctacattggcatgcaggatcaatggtacagcttcaatatgtttgatgcccaagcctggtatgcccgtgatgtgattatgggtcgactgccattgccatcaaaagaagagatgaaagccgacagcatggcctggcgtgaaaaagaactgacgctggttacggctgaagaaatgtacacctaccagggtgactacattcagaatctgattgatatgactgactatccgtcatttgatattccggcaaccaacaaaactttcctggaatggaaacatcacaaaaaagaaaacatcatgactttccgtgaccactcataccgttcactgatgactggcacgatggcaccgaaacatcacacaccatggatagatgcactggatgattctctggaagcctatctctctgataagagcgaaattcctgtggctaaagaagcttaa

SEQ ID NO: 3 - DNA, 5904 bp, Corynebacterium glutamicum
  source 1..5904: mol_type = genomic DNA; strain = ATCC 13032
atggacaatggcatgacaatcaccacagaacattcaactcatcctgatcttgatttcaatgatgagatttatcgggaactaaaccgcatctgcgcttcgctatctcaacagtgcagcacatatcaaccagagttccgtacctgcctagatgctgctttccaagctttgcgaggtggcaagttaatccgccctcgaatgctactggggctatacaacacgcttgtagacgatgacattgaggtcaaactcaacaccgttttacaggtagcagtggctttagaactactgcatttttcccttttggttcatgacgatgttattgacggagacctctatcgccgaggcaaacttaattttattgggcagattctcatgcatcgcacacctgaaagttttgcacaaatccagcgcgatccagagcatctagattgggcacaatctaatggactgcttatgggaaatctttttcttgctgccacccatcaaatcttcgcgcgccttgaccttccacatcaccaacgggttcgacttttagatttactcaaccacacgataaatgacactattgtgggtgagtttcttgatgtgggattaagcagcaaagccatcagccccaatatggacattgctctagaaatgagtcggctaaaaacagccacatacacttttgaacttccaatgagagcagcggcaattctcgcggaactacctcaggagattgaaacaaagataggtgagataggcacaaacttgggcatcgcttatcaattgcaggacgattacttatctacttttggtgacgcagccgaacacggcaaagatgccttttctgaccttcgagaaggaaaagaaactacaattatcgccttcgctcgagatactgctaaatggactgatattcaagacaacttcggctccgcagatctgagcacctctcaggcagagcgaattcaacatcttctcatacagtgtggagcaaagaatcactccttgaatgccatctccgaccacttaaatatctgccgttcgatgatcaaaacactaagcccccaggtagatcccaaggctcaaaatttattacttaaacaagttgagcaactagccagccgcaaatcttagaactaacctttacgcctttaacggaagtgaatttgatgtctactagcatcacaacagagaacaagaagaaatctggtcctcctcgcttgatgagaatctttctgcccgccttgctaattttagtttggcttgtaggagctggagtcggcggtccttattttggcaaggttagtgaggtctcctccaacagccagaccacatatctgccagaatctgccgatgccactcaagtacaggaacagttgggagattttactgattctgaatccatcccagccattgtcgtaatggtcagcgatgaacccttaacacagcaagacatcacacaactcaatgaagttgttgctgggctttcagaattagacatagtttccgatgaagtctcccctgctattccatccgaggacggcagagctgtccaagtgtttgtccccctcaatccatcagcggagctgacggaaagcgtcgagaagctctctgagaccttgacccagcaaacgccggactatgtgagcacctatgtgaccggaccggctgggtttaccgctgatctcagcgcagctttcgcgggtattgatgggctactcctagcagtcgccttggctgccgtccttgtcattcttgtcatcgtctatcgctccttcattctgcccatcgccgtgcttgccaccagtttgtttgcgctgactgtagctctattggtggtgtggtggctagctaagtgggacatcctgctgctttcgggtcagactcaaggcatcctcttcattctggtcattggcgccgccaccgactactcattgctatacgttgctcgtttccgtgaagagttacgcgttcaacaagataaagggatagccacagggaaagccatccgggcatcggtggaacccattcttgcctcgggcagcactgttattgcgggcctcctttgtttgctatttagtgatttgaaatctaactccacgctaggtccagtagcttcggtgggcattatttttgcaatgctttctgctcttactctgctaccagccctgctgtttgtattcggtcgggtggccttttggcccaagcgaccaaaatacgaacctgaaaaagcccgtgcgaaaaacgacatccccgccagcgggatctggtcaaaagtggctgatttagtggagcagcatcctcgtgcaatctgggtatctacacttattgtgcttctcttgggtgcggctttcgttcccacactaaaagcggacggtgtgtcccaatccgacctagttctgggttcctctgaagcacgtgatggccagcaggctttaggcgaacacttccccggtggatccggcagtcctgcttatattatcgttgatgaaacacaggcagcacaggctgctgacgtagtccttaacaacgacaatttcgagactgtaactgtaactagtgctgactccccctctggctcagccccaatcaccgctgacggtattgtgccgttaggttctggtacagctccaggcccggtagttgtagaagggcaagtccttttacaagcaacacttgtcgaagcaccagattccgaagaagctcaaaaagctattcgcagtatccgccaaacttttgcagatgaaaatatatcagcggtagtaggcggtgtcactgcaacttccgtagacactaacgatgcctccatccatgaccgcaacctgatcatcccaattgtattgctggtcattttggttattctcatgctgttgctgcggtctattgtcgcaccactcctgctagtagtcaccaccgtggtgtcttttgctactgctttaggcgtggctgctttacttttcaatcacgttttcagtttcccaggagcagaccccgcagtacctctctacggatttgtatttttagtagccttgggcatcgactacaacattttcttagtcacccgaatccgtgaagaaaccaaaacccacggcacaagacttggaattcttcgaggcctgacagtaaccggcggagtaattacctcagctggagtagttctcgccgcaacgttcgcagcactctatgtcatcccaattctattcctggcacaaattgccttcattgtcgcttttggagttcttattgataccctgctcgttcgcgccttcttggtgcctgctttgttctacgacatcggaccgaaaatctggtggccgtcaaaattgtccaatcagaaataccagaagcagcctcagctatgacacaccaaaattcgcctctcttccttaaaagtgcactgagactttacaatcgggcctcattcaaggcttcacataaagtgatcgaagaatattcgacgagcttcagtctgtctacgtggttgctatccccacgcatacgaaatgacatacgaaatctctatgcagtagttcgtatcgccgatgagattgtcgacggcactgcacatgccgctggttgctcaactgccaaaatcgaagagattctcgatgcctatgaaattgcggttcttgcagcaccacaacaacgcttcaacacagatcttgttttacaagcttatggtgaaactgcccgacgctgtgatttcgaacaagagcatgtaatagccttctttgcatcaatgcgtaaggacctcaaagctaatacacacgacccagatagcttcacaacgtatgtctatggctccgcggaagttataggcctgctttgtctcagcgttttcaaccaaggtagaacgattagcaaaaaacggctagagattatgcaaaacggagcccgctcattgggagcggcattccagaaaattaactttctccgtgacttggcagaagatcagcaaaatttgggccgattttatttccccaaaaccagccaaggaactcttactaaagaacaaaaagaagatctcatcgctgatatccgtcaagacctagcaattgcccacgatgcatttccagaaataccagtgcaggctcgcatcggagtgatctctgcttatttgctctttcaaaaactcactgaccgaattgaggctactcctaccgccgatttattgcgggagcgaatcagagttccacttcatatcaaactctctacactcgctagagccacgatgaaaggtctatctatgagcatctacagaaagaattcgtgatgaaggtctcgactaaaactccacgctcctcaggtaccgccgtagtcataggcgcaggtgttgctggtttagccacttctgcacttttagcacgtgatggctggcaagtaactgttttggaaaaaaatactgatgtcggtggccgagctggatcgcttgaaatatcaggctttcctggctttcgatgggataccggaccttcttggtacctcatgcccgaggcctttgaccatttcttcgcactttttggtgcatgtacttctgattatctcgatttggtagaattaacgcctggttatcgagttttttctggcacacatgacgctgtcgatgtccccactgggcgtgaagaagcaattgcgctattcgaatccatcgaacccggcgcgggtgcaaaactaggaaattatcttgatagcgcggcagacgcctatgacattgccattgatagattcctttataataatttctccacgttaggcccgctgcttcaccgggatgtactgacccgagctggccgactgttttctctactgacccgttctttacaaaagtacgtaaatagtcaattcagtagcccggtgttgcgccagatcctaacctatccagcagtcttcctgtcttcccgacccactactaccccatcgatgtaccacttgatgagtcataccgatttggtgcagggagtgaaataccctataggtggttttactgcagtggttaacgctctgcatcagttagcgctggaaaacggggttgagtttcaactcgattctgaggtcatttccatcaacactgcttcatcgaggggcaacacaagcgccacaggtgtgagcttgcttcacaacagaaaagtgcaaaatctagatgcggatcttgtggtttcagcaggcgacctacaccatacagaaaataatctgcttccccgggaacttcgaacctatcccgaacgatattggtccaatcgcaatcctggaattggagcggtattaatcctcctgggcgtaaaaggagagttaccccagctcgaccatcacaaccttttcttcagtgaagattggacagatgattttgctgtagttttcgacgggcctcaacttacccgcccccacaatgcatcaaattccatttatgtctccaagccttcaacgtccgaagacggcgttgcacctgctggatacgaaaacctttttgttttaattccgaccaaggcctctagcagcatcggccacggtgatgcgtatatgcagtcggcttcagcatccgtggaaacaatcgcgtcacatgcaatcaatcaaattgctacgcaagccggcatccctgacctcactgaccgaattgtggtcaaacgcaccattggccctgcggattttgagcaccgctaccattcatgggtaggcagtgcgctgggtccagcacataccctcagacagtccgctttcttaagagggcgcaatagctcccgcaaggtcaataacctcttctattccggtgccaccaccgtcccgggtgtaggaatacccatgtgtttaatttctgccgagaatattattaagcgtttacatgccgataccagtgcaggaccactgcccgaaccattgccgcctaaaacgacaccatctcaaaagacctcatacgatcattaa

SEQ ID NO: 4 - DNA, 1686 bp, Arabidopsis thaliana
  source 1..1686: mol_type = genomic DNA
atggcgccacaagaacaagcagtttctcaggtgatggagaaacagagcaacaacaacaacagtgacgtcattttccgatcaaagttaccggatatttacatcccgaaccacctatctctccacgactacatcttccaaaacatctccgaattcgccactaagccttgcctaatcaacggaccaaccggccacgtgtacacttactccgacgtccacgtcatctcccgccaaatcgccgccaattttcacaaactcggcgttaaccaaaacgacgtcgtcatgctcctcctcccaaactgtcccgaattcgtcctctctttcctcgccgcctccttccgcggcgcaaccgccaccgccgcaaaccctttcttcactccggcggagatagctaaacaagccaaagcctccaacaccaaactcataatcaccgaagctcgttacgtcgacaaaatcaaaccacttcaaaacgacgacggagtagtcatcgtctgcatcgacgacaacgaatccgtgccaatccctgaaggctgcctccgcttcaccgagttgactcagtcgacaaccgaggcatcagaagtcatcgactcggtggagatttcaccggacgacgtggtggcactaccttactcctctggcacgacgggattaccaaaaggagtgatgctgactcacaagggactagtcacgagcgttgctcagcaagtcgacggcgagaacccgaatctttatttccacagcgatgacgtcatactctgtgttttgcccatgtttcatatctacgctttgaactcgatcatgttgtgtggtcttagagttggtgcggcgattctgataatgccgaagtttgagatcaatctgctattggagctgatccagaggtgtaaagtgacggtggctccgatggttccgccgattgtgttggccattgcgaagtcttcggagacggagaagtatgatttgagctcgataagagtggtgaaatctggtgctgctcctcttggtaaagaacttgaagatgccgttaatgccaagtttcctaatgccaaactcggtcagggatacggaatgacggaagcaggtccagtgctagcaatgtcgttaggttttgcaaaggaaccttttccggttaagtcaggagcttgtggtactgttgtaagaaatgctgagatgaaaatagttgatccagacaccggagattctctttcgaggaatcaacccggtgagatttgtattcgtggtcaccagatcatgaaaggttacctcaacaatccggcagctacagcagagaccattgataaagacggttggcttcatactggagatattggattgatcgatgacgatgacgagcttttcatcgttgatcgattgaaagaacttatcaagtataaaggttttcaggtagctccggctgagctagaggctttgctcatcggtcatcctgacattactgatgttgctgttgtcgcaatgaaagaagaagcagctggtgaagttcctgttgcatttgtggtgaaatcgaaggattcggagttatcagaagatgatgtgaagcaattcgtgtcgaaacaggttgtgttttacaagagaatcaacaaagtgttcttcactgaatccattcctaaagctccatcagggaagatattgaggaaagatctgagggcaaaactagcaaatggattgtga

SEQ ID NO: 5 - DNA, 1209 bp, Oryza sativa
  source 1..1209: mol_type = genomic DNA
atggcaccgacgacgaccatgggaagcgccttatacccgctcggcgaaatgcggcggtcgcagcgcgccgacgggctcgccgccgtgctcgccatcggcaccgccaaccctcccaactgcgttacccaggaggagatccccgacttctacttccgcgtcaccaacagcgaccacctcactgccctcaaggacaagttcaagcgtatctgtcaggaaatgggcgtgcagaggcggtacctgcaccacacggaggagatgctgtccgcacacccggagttcgtggaccgcgacgcgccgtcgctcgacgcgcggctggacatcgccgcggacgccgtgccggagctggcggcggaggccgccaagaaggcgatcgccgagtggggccgcccggccgccgacatcacccacctcgtcgtcaccaccaactccggcgcccacgtcccgggtgtcgacttccgcctcgtcccgctcctcggcctccgcccctccgtgcgccgcaccatgctccacctcaacggctgcttcgccggctgcgccgcgctccgcctcgccaaggacctcgccgagaacagccgcggcgcgcgcgtcctcgtcgtcgccgccgagctcaccctcatgtacttcaccgggcccgacgagggctgcttcaggacgctcctcgtccagggcctgttcggcgacggcgcggccgccgtcattgtcggcgccgacgccgacgacgtcgagcgcccgctgttcgagatagtgtcggcggcgcagacgatcatcccggagtcggaccacgccctgaacatgcggttcacggagcgccgcttggacggcgtcctcgggcggcaggtcccggggctcatcggggacaacgtcgagcggtgcctcctggacatgttcggcccacttctcggcggcgacggcggcggcgggtggaacgacctgttctgggcggtgcacccgggctcgtcgacgatcatggaccaggtcgacgcggcgctcggcctcgagcccggcaagctggcggcgagccgccgtgtgctcagcgactacgggaacatgtctggcgctacggtgatcttcgcgctggacgagctgcgccggcagcgcaaggaggcggcggcggcgggtgaatggccggagttgggagtgatgatggcgttcggcccggggatgaccgttgatgcgatgctgttgcacgccacctctcatgtgaattaa

(19) United States
(12) Patent Application Publication
(10) Pub. No.: US 2026/0071242 A1
(43) Pub. Date: Mar. 12, 2026

(54) METHOD TO PRODUCE COLORED BIOPLASTICS USING MICROBES

(71) Applicant: Angela Zhan, North Logan, UT (US)

(72) Inventor: Angela Zhan, North Logan, UT (US)

(73) Assignee: Angela Zhan, North Logan, UT (US)

(21) Appl. No.: 18/447,262

(22) Filed: Aug. 9, 2023

(51) Int. Cl. C12P 7/625; C12N 9/00; C12N 9/02; C12N 9/04; C12N 9/10; C12N 15/52; C12N 15/70

(52) U.S. Cl. CPC C12P 7/625; C12N 9/0006; C12N 9/001; C12N 9/0073; C12N 9/1029; C12N 9/1085; C12N 9/93; C12N 15/52; C12N 15/70; C12Y 101/01036; C12Y 103/05005; C12Y 114/13148; C12Y 203/01016; C12Y 205/01001; C12Y 205/01032; C12Y 602/01012

(57) ABSTRACT

The present invention introduces an innovative approach for the manipulation of microorganisms to generate colored bioplastics. This is achieved by concurrently expressing genes responsible for pigment production and genes involved in bioplastic synthesis within a microbial host. These genes can be synthesized, obtained from a different host through cloning, or naturally occurring within the host organism. The resultant color compounds become encapsulated within the extended bioplastic polymers within the cells, resulting in the formation of naturally pigmented bioplastics.

### INCORPORATION BY REFERENCE

The DNA sequences discussed in this patent application are incorporated by reference to the Sequence Listing file, named “patentsequencelisting,” which was submitted with the current application. This Sequence Listing file was created on Jul. 20, 2024 and its size is 19,000 bytes.

### REFERENCE TO A “SEQUENCE LISTING”

The nucleotide sequence of a representative polyhydroxybutyrate biosynthetic gene cluster phaCAB is shown in sequence listing as SEQ ID No: 1. The nucleotide sequence of a representative indole 3-hydroxylase gene fmo is shown in sequence listing as SEQ ID No: 2. The nucleotide sequence of a representative lycopene biosynthetic gene cluster containing fragment crtE-cgl0617-crtB-crt/is shown in sequence listing as SEQ ID No: 3. The nucleotide sequence of a representative 4-coumarate: coenzyme A ligase gene 4cl is shown in sequence listing as SEQ ID No: 4. The nucleotide sequence of a representative curcuminoid synthase gene cus is shown in sequence listing as SEQ ID No: 5.

### BACKGROUND

Plastics have permeated nearly every facet of our daily existence. A substantial portion of these plastics originates from petroleum, rendering them resistant to degradation and instigating severe ecological predicaments. For example, it takes about 450 years to degrade a plastic water bottle in the environment. Furthermore, the rapid depletion of fossil fuel reserves, coupled with their non-renewable nature, exacerbates these concerns. By contrast, bioplastics are derived from renewable biomass sources and thus are environmentally friendly. However, bioplastics are dyed with synthetic dyes (causing environmental pollution) to afford various colors for different products. It is desirable to make colored bioplastics in a natural and sustainable way.

Polyhydroxybutyrate (PHB) is a bio-derived and biodegradable plastic (FIG. 1) from bacteria, which stands as a compelling alternative to conventional petroleum-based plastics. Natural pigments are compounds generated by living organisms, such as plants, animals, and microorganisms. Some well-known natural pigments are lycopene (red), indigo (blue), and curcumin (yellow), as shown in FIG. 2. Many of these color compounds are poorly water-soluble or water-insoluble. The foundational premise of this invention lies in the approach to engineer microorganisms to synthesize water-sparingly soluble or water-insoluble pigments within the same cellular environment as PHB, transforming these pigments into captives of the polymeric PHB structure during their joint biosynthetic journey. This orchestrated process culminates in the generation of naturally colored bioplastics.

Carotenoids are a group of natural pigments with health-benefiting activities. Lycopene is a natural bright red carotenoid pigment found in both photosynthetic and non-photosynthetic organisms. Curcumin is a yellow dye from turmeric, which has been used as a food colorant and dietary supplement. Indigo is a natural dye extracted from the leaves of several plants of the Indigofera genus and shows a bright blue color. All these above-mentioned dyes are either insoluble in water or have extremely low water solubility.

The current invention introduces a novel technique for generating colored bioplastics by amalgamating the PHB biosynthetic route with a pigment-forming pathway within a microbial host, such as Escherichia coli. Several illustrative instances are furnished within this patent application, including co-expressing the PHB biosynthetic genes with the pathway for lycopene synthesis, indigo formation, or curcumin biosynthesis, yielding PHB materials exhibiting corresponding colors. This innovative colored bioplastic material can be directly incorporated into the production of an array of items, including but not limited to utensils and toys, obviating the necessity for chemical dyeing. This methodology extends to the utilization of PHB and other naturally occurring pigments, thus enabling the creation of vibrant bioplastics.

### SUMMARY

A novel method has been devised to empower bacteria in the creation of colored bioplastics. This method involves the concurrent expression of PHB-producing genes alongside pigment-producing genes within a microbial host. Upon their synthesis, pigments exhibiting either poor water solubility or insolubility undergo rapid precipitation. Subsequently, they are entrapped within the polymeric structure of PHB, effectively producing colored bioplastic material within the cellular matrix. This methodology is extensible across various microbial species and lends itself to the production of diverse-hued bioplastics.

### BRIEF DESCRIPTION OF THE DRAWINGS

FIG. 1 illustrates the structures of polyhydroxybutyrate and three natural pigments including lycopene, indigo, and curcumin.

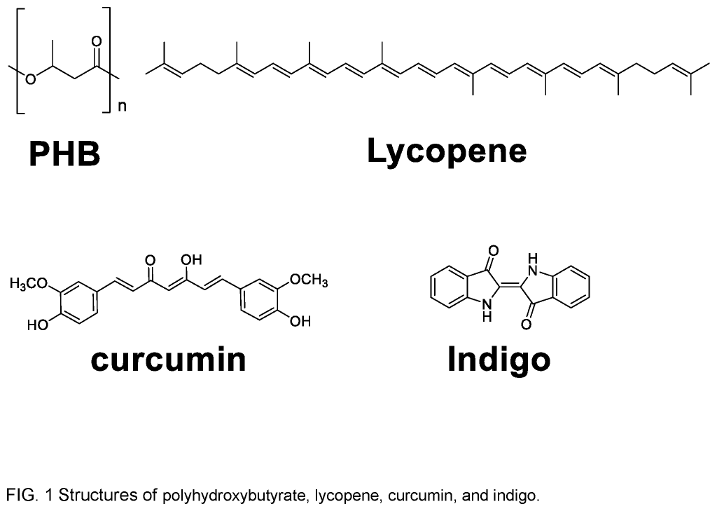

FIG. 2 shows the maps of plasmids constructed to produce colored polyhydroxybutyrate materials in E. coli.

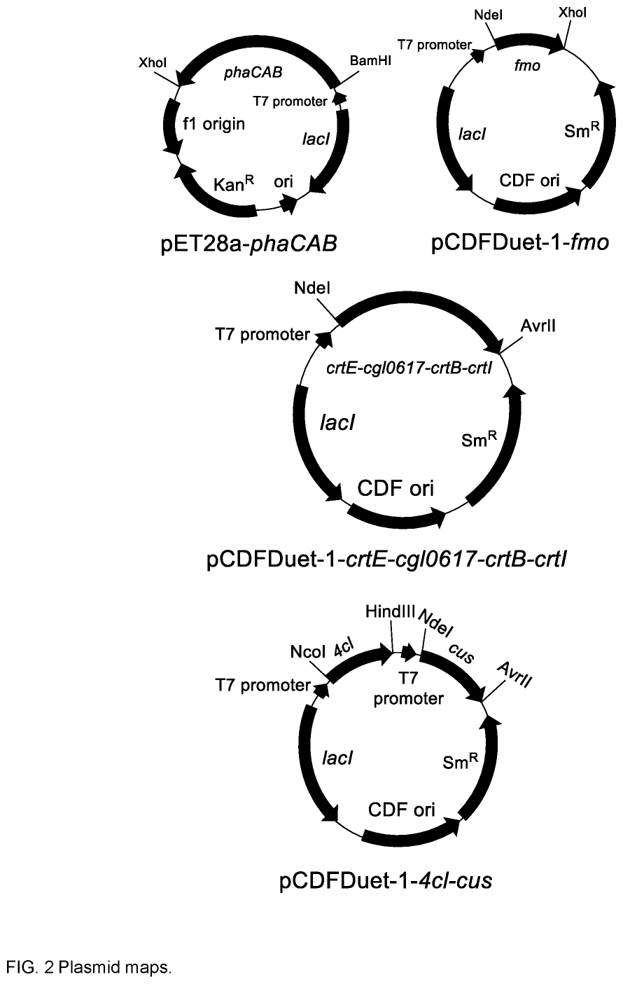

### DETAILED DESCRIPTION

The scope of this disclosure encompasses methodologies for creating engineered strains capable of producing colored bioplastics. Throughout the ensuing exposition, comprehensive elucidation of specific preferred embodiments is provided to facilitate a comprehensive understanding. However, it is to be noted that those proficient in the relevant field will discern that embodiments can be executed with the omission of specific details, incorporation of alternative methods, components, materials, etc. Occasionally, commonplace structures, materials, or procedures may not be exhaustively delineated to prevent obfuscation of salient aspects of the preferred embodiments. Moreover, the delineated attributes, genes, structures, strains, or traits are amenable to amalgamation in diverse configurations across a spectrum of alternative embodiments. Thus, the ensuing elaboration, which furnishes additional intricacies of the present invention's embodiments, as exemplified in certain aspects within the illustrations, is not intended as a delimiting factor but rather serves as a representative depiction of the manifold embodiments of the invention.

Throughout this specification and the ensuing claims, the use of singular forms like “a,” “an,” and “the” encompasses their plural counterparts, unless contextual clarity dictates otherwise. All disclosed ranges encompass, unless explicitly stated, both endpoints and the intermediary values within. Furthermore, instances of “optional” or “optionally” pertain to scenarios wherein subsequent circumstances may or may not transpire, encompassing occurrences as well as non-occurrences of said circumstances. Similarly, the phrases “one or more” and “at least one” denote situations wherein one or more of the subsequently mentioned circumstances may manifest, encompassing both singular occurrences and multiple occurrences of those circumstances.

In a specific embodiment, this disclosure furnishes techniques for genetically engineering bacteria to fabricate diverse bioplastics imbued with specified colors. By way of example, the present disclosure outlines the co-expression of PHB biosynthetic genes with pigment-producing genes in E. coli BL21 (DE3), resulting in the creation of red, blue, or yellow bioplastics. The methods described herein generally provide for a method to enable bacteria to produce colored bioplastics in an environmentally-friendly and sustainable manner. The details for the procedure are provided.

The present disclosure also provides for the methods to co-express PHB- and pigment-producing genes in bacteria.

The genes encoding the PHB- and pigment-producing genes can be directly amplified from the genome or cDNA of a related microbial strain or synthetically constructed. The genes may be modified for improved activity or expression.

Expression of the PHB- and pigment-producing genes can be achieved in various hosts such as E. coli, Bacillus subtilis, Pseudomonas putida, and other microbial strains. Any suitable bacterial strain, vector or culture condition may be used for the expression of PHB- and pigment-producing genes to produce colored bioplastics. As an illustration, E. coli BL21 (DE3) represents a suitable bacterial strain. Alternatively, any species or strain of E. coli may be used. Broadly, a suitable microbial strain is any bacterial strain capable of functionally co-expressing PHB- and pigment-producing genes. In some embodiments, colored bioplastics may be generated by a microbial strains harboring a vector or vectors harboring the PHB- and pigment-producing genes. The vector or vectors may be plasmids.

Bacteria harboring the PHB- and pigment-producing genes may manufacture colored bioplastics from exogenously supplied substrates such as L-tryptophan, indole, and ferulic acid. Alternatively, colored bioplastics may be synthesized by bacteria containing the PHB- and pigment-producing genes without necessitating the influx of aforementioned substrates.

Engineered microbial strains harboring PHB- and pigment-producing genes are cultivated within an appropriate medium. In instances featuring an inducible promoter within the vector, a specific inducer is introduced into the culture to trigger protein expression. In cases utilizing a constitutive promoter, the introduction of an inducer is not required. To engender colored bioplastics, for instance, blue PHB, a substrate such as indole and tryptophan is supplemented to the culture when the engineered strains lack innate substrate production capacity. Conversely, if the bacterial hosts are intrinsically proficient in generating essential substrates, no external substrates are necessary.

In conclusion, this innovation provides a method for constructing bacterial strains to produce naturally colored bioplastics. The ensuing examples are illustrative in nature and should not impose constraints on the disclosure. Those versed in the field would readily identify established techniques and conditions for the cloning or synthesis of PHB- and pigment-producing genes, the expression of these genes within bacteria, and the assessment of colored PHB production. Each of these varied embodiments falls within the scope of this invention.

### EXAMPLES

The following materials and methods may be used in carrying out the various embodiments of the invention.

### Example 1. Bacterial Strains, Vectors, and Culture Conditions

Ralstonia eutropha ATCC 17699 and Corynebacterium glutamicum ATCC 13032 were from the American Type Culture Collection. R. eutropha ATCC 17699 was grown in nutrient-rich medium containing 1% polypeptone, 1% yeast extract, 0.5% beef extract, and 0.5% (NH₄)₂SO₄ at 30° C. C. glutamicum ATCC 13032 was grown in tryptic soy broth containing 1.7% tryptone, 0.3% soytone, 0.25% dextrose, 0.5% sodium chloride, and 0.25% dipotassium phosphate at 37° C. E. coli XL1-Blue and BL21 (DE3) were purchased from Agilent. Both E. coli strains were routinely grown at 37° C. in LB medium. For colored bioplastics production, the engineered E. coli strains were cultured at 30° C. in LB medium with appropriate antibiotic(s) (kanamycin, 50 μg/mL; streptomycin, 50 μg/mL) after protein expression induction.

### Example 2. DNA Manipulations

The genomic DNAs of R. eutropha ATCC 17699 and C. glutamicum ATCC 13032 were isolated using a GeneJET™ Genomic DNA Purification Kit (Thermo Fisher). Plasmids in E. coli were extracted using a GeneJET™ Plasmid Miniprep Kit (Thermo Fisher). E. coli strains were transformed with plasmids through heat shock. Phusion high-fidelity DNA polymerase, T4 DNA ligase, and restriction enzymes were purchased from New England Biolabs.

### Example 3. Construction of PHB-Producing Plasmid in E. coli

A 3,851-bp gene fragment was amplified from the genomic DNA of R. eutropha ATCC 17699, which contains the phaC, phaA, and phaB genes, using a pair of specific primers including 5′-ATCGGGATCCATGGCGACCGGCAAAGGCG-3′ and 5′-ATCGCTCGAG TCAGCCCATATGCAGGCCGCCGT-3′ via PCR. Phusion high-fidelity DNA polymerase from New England Biolabs was used for PCR. The PCR product was digested with BamHI and XhoI and then gel recovered. The gene fragment was ligated into pET28a between the BamHI and XhoI sites using T4 DNA ligase. The ligation product was transferred into E. coli XL1-Blue through chemical transformation and correct colonies were selected on a LB+kanamycin agar plate.

The colonies were picked and grown in LB broth supplemented with kanamycin at 37° C. with shaking at 250 rpm overnight. Plasmids were extracted from the cultures and subjected to digestion check with BamHI and XhoI to identify the correct plasmid pET28a-phaCAB.

### Example 4. Construction of Indigo-Producing Plasmid in E. coli

The fmo gene from Methylophaga aminisulfidivorans MP 54_1 encodes an indigo 3-hydroxylase. The gene was synthesized by a commercial company with NdeI and XhoI on the 5′ and 3′ sides, respectively. The gene was digested with NdeI and XhoI and then the gel-recovered gene was ligated into the pCDFDuet-1 vector between the same sites using T4 DNA ligase. The ligation product was transferred into E. coli XL1-Blue and successful transformants were selected on a LB+streptomycin agar plate.

The colonies were picked and grown in LB broth supplemented with streptomycin at 37° C. with shaking at 250 rpm overnight. Plasmids were extracted from the cultures and subjected to digestion check with NdeI and XhoI to identify the correct plasmid pCDFDuet-1-fmo.

### Example 5. Construction of Lycopene-Producing Plasmid in E. coli

A 5,904-bp gene fragment was amplified from the genomic DNA of C. glutamicum ATCC 13032 via PCR using a pair of specific primers including 5′-ATCGCATATGGACAATGGCATGACAATC-3′ and 5′-ATCGCCTAGGTTAATGATCGTATGAGGTC-3′. Phusion high-fidelity DNA polymerase from New England Biolabs was used for PCR. This fragment contains crtE, cgl0617, crtB and crtl that encode geranylgeranyl pyrophosphate synthetase, multidrug efflux protein, phytoene synthase, and phytoene desaturase, respectively. The crtE, crtB and crtl genes are required for lycopene biosynthesis from farnesyl diphosphate (FPP). The PCR product was digested with NdeI and AvrII. The gel-recovered fragment was ligated into pCDFDuet-1 between the NdeI and AvrII sites using T4 DNA ligase. The ligation product was transferred into E. coli XL1-Blue and successful transformants were selected on a LB+streptomycin agar plate.

The colonies were picked and grown in LB broth supplemented with streptomycin at 37° C. with shaking at 250 rpm overnight. Plasmids were extracted from the cultures and subjected to digestion check with NdeI and Avril to identify the correct plasmid pCDFDuet-1-crtE-cgl0617-crtB-crtl.

### Example 6. Construction of Curcumin-Producing Plasmid in E. coli

The 4-coumarate: coenzyme A ligase gene 4cl from Arabidopsis thaliana and the curcuminoid synthase gene cus from Oryza sativa were synthesized by a commercial company. The synthesized 4cl gene was flanked by the NcoI and HindIII sites, and cus was flanked by the NdeI and AvrII sites. The 4cl gene was digested with NcoI and HindIII and the gel-recovered gene was ligated into pCDFDuet-1 between the same sites using T4 DNA ligase. The ligation product was transferred into E. coli XL1-Blue and successful transformants were selected on a LB+streptomycin agar plate.

The colonies were picked and grown in LB broth supplemented with streptomycin at 37° C. with shaking at 250 rpm overnight. Plasmids were extracted from the cultures and subjected to digestion check with NcoI and HindIII to identify the correct plasmid pCDFDuet-1-4cl.

The cus gene was digested with NdeI and Avril and the gel-recovered gene was ligated into pCDFDuet-1-4cl between the NdeI and Avril sites using T4 DNA ligase. The ligation product was transferred into E. coli XL1-Blue and successful transformants were selected on a LB+streptomycin agar plate.

The colonies were picked and grown in LB broth supplemented with streptomycin at 37° C. with shaking at 250 rpm overnight. Plasmids were extracted from the cultures and subjected to digestion check with NdeI and Avril to identify the correct plasmid pCDFDuet-1-4cl-cus.

### Example 7. Production of Blue PHB in E. coli BL21 (DE3)

E. coli BL21 (DE3) was transformed with pET28a-phaCAB and pCDFDuet-1-fmo through chemical transformation. The correct transformants were selected on LB agar supplemented with both kanamycin and streptomycin. A colony of E. coli BL21 (DE3)/pET28a-phaCAB+pCDFDuet-1-fmo was picked into a 250-mL flask containing 50 mL of LB broth supplemented with kanamycin and streptomycin. The flask was incubated at 37° C. with shaking at 250 rpm until the OD₆₀₀ value reached 0.4-0.6. The inducer, isopropyl-β-D-1-thiogalactopyranoside (IPTG) was then added into the culture at a final concentration of 200 UM and 5 mg of indigo was supplemented as the substrate. The culture was maintained at 30° C. with shaking at 250 rpm for 24 hours.

The cells were harvested by centrifugation at 3,500 rpm for 10 min. The cells were resuspended in 10 mL of chloroform. After 8 minutes of sonication, the cell lysate was centrifuged at 15,000 rpm for 10 minutes. The supernatant was collected in a round-bottom flask and dried on a rotavapor under reduced pressure. The blue PHB polymer product was seen on the bottom of the flask.

### Example 8. Production of Red PHB in E. coli BL21 (DE3)

E. coli BL21 (DE3) was transformed with pET28a-phaCAB and pCDFDuet-1-crtE-cgl0617-crtB-crt/through chemical transformation. The correct transformants were selected on LB agar supplemented with both kanamycin and streptomycin. A colony of E. coli BL21 (DE3)/pET28a-phaCAB+pCDFDuet-1-crtE-cgl0617-crtB-crt/was picked into a 250-mL flask containing 50 mL of LB broth supplemented with kanamycin and streptomycin. The flask was incubated at 37° C. with shaking at 250 rpm until the OD₆₀₀ value reached 0.4-0.6. The inducer IPTG was then added into the culture at a final concentration of 200 μM. The culture was maintained at 30° C. with shaking at 250 rpm for 24 hours.

The cells were harvested by centrifugation at 3,500 rpm for 10 min. The product was extracted from the cells by sonication using chloroform. The solvent was evaporated on a rotavapor and the red PHB polymer product was seen on the bottom of the flask.

### Example 9. Production of Yellow PHB in E. coli BL21 (DE3)

E. coli BL21 (DE3) was transformed with pET28a-phaCAB and pCDFDuet-1-4cl-cus through chemical transformation. The correct transformants were selected on LB agar supplemented with both kanamycin and streptomycin. A colony of E. coli BL21 (DE3)/pET28a-phaCAB+pCDFDuet-1-4cl-cus was picked into a 250-mL flask containing 50 mL of LB broth supplemented with kanamycin and streptomycin. The flask was incubated at 37° C. with shaking at 250 rpm until the OD₆₀₀ value reached 0.4-0.6. The inducer IPTG was then added into the culture at a final concentration of 200 μM and 10 mg of ferulic acid was supplemented as a substrate. The culture was maintained at 30° C. with shaking at 250 rpm for 24 hours.

The cells were harvested by centrifugation at 3,500 rpm for 10 min. The product was extracted from the cells by sonication using chloroform. The solvent was evaporated on a rotavapor and the yellow PHB polymer product was seen on the bottom of the flask.

It should be recognized that numerous features, functions, and alternatives as disclosed above, along with others, can be advantageously integrated into various diverse systems or applications. Additionally, unforeseen or unanticipated alternatives, modifications, variations, or enhancements may be later devised by individuals skilled in the relevant field, all of which are intended to fall within the scope of the ensuing claims.

### NON-PATENT CITATIONS

[1] Kim M J, Noh M H, Woo S, Lim H G, Jung G Y. Enhanced lycopene production in Escherichia coli by expression of two MEP pathway enzymes from Vibrio sp. Dhg. Catalysts 2019, 9, 1003.
[2] Wu J, Chen W, Zhang Y, Zhang X, Jin J-M, Tang S-Y. Metabolic engineering for improved curcumin biosynthesis in Escherichia coli. Journal of Agricultural and Food Chemistry 2020, 68 (39), 10772-10779.
[3] Han G H, Shin H-J, Kim S W. Optimization of bio-indigo production by recombinant E. coli harboring fmo gene. Enzyme and Microbial Technology 2008, 42 (7), 617-623.
[4] Heider S A, Peters-Wendiech P, Wendisch V F. Carotenoid biosynthesis and overproduction in Corynebacterium glutamicum. BMC Microbiology 2012, 12, 198.
[5] Katsuyama Y, Matsuzawa M, Funa N, Horinouchi S. In vitro synthesis of curcuminoide by type III polyketide synthase from Oryza sativa. Journal of Biological Chemistry 2007, 282 (52), 37702-37709.

What is claimed is:

1. A method for the production of colored bioplastics, comprising the following steps:
(a) Acquiring polyhydroxybutyrate biosynthetic genes by means of gene synthesis, PCR amplification, or genomic DNA digestion;
(b) Obtaining pigment-forming gene(s) through gene synthesis, PCR amplification, or genomic DNA digestion; and
(c) Co-expressing the polyhydroxybutyrate biosynthetic genes and pigment-forming gene(s) within a bacterial host to yield colored bioplastics.

2. The method of claim 1 further comprises using natural, recombinant, or synthesized polyhydroxybutyrate biosynthetic genes that encode a beta-ketothiolase, acetoacetyl-coenzyme A reductase, and polyhydroxybutyrate synthase, respectively.

3. The method of claim 1 further comprises using a natural, recombinant, or synthesized indole 3-hydroxylase gene.

4. The method of claim 1 further comprises using natural, recombinant, or synthesized lycopene-forming genes that encode geranylgeranyl pyrophosphate synthetase, phytoene synthase, and phytoene desaturase, respectively.

5. The method of claim 1 further comprises using natural, recombinant, or synthesized curcumin-forming genes that encode 4-coumarate: coenzyme A ligase and curcuminoid synthase, respectively.

6. The method of claim 1, further incorporating an expression system for polyhydroxybutyrate biosynthetic genes and pigment-forming genes using either an inducible or constitutive promoter within a bacterial host; said expression being achievable through an expression plasmid or integration of the respective halogenase gene into the bacterial host's genome.
